(19) United States
(12) Patent Application Publication
(10) Pub. No.: US 2025/0253047 A1
(43) Pub. Date: Aug. 7, 2025

(54) BIOMARKERS FOR IDIOPATHIC PULMONARY FIBROSIS AND METHODS OF PRODUCING AND USING SAME

(71) Applicant: Siemens Healthcare Diagnostics Inc., Tarrytown, NY (US)

(72) Inventors: Andrew Beard, Princeton, NJ (US); Chris Green, Pleasanton, CA (US); Sebastian Kronmueller, San Francisco, CA (US)

(73) Assignee: Siemens Healthcare Diagnostics Inc., Tarrytown, NY (US)

(21) Appl. No.: 18/855,802

(22) PCT Filed: Apr. 19, 2023
(86) PCT No.: PCT/US2023/065978

§ 371 Date: Oct. 10, 2024

Related U.S. Application Data
(60) Provisional application No. 63/363,282, filed on Apr. 20, 2022.

(51) Int. Cl. G16H 50/20; G01N 33/53; G01N 33/536; G01N 33/68; G16H 20/10; G16H 20/40

(52) U.S. Cl. CPC G16H 50/20; G01N 33/5308; G01N 33/536; G01N 33/6893; G16H 20/10; G16H 20/40; G01N 2333/78; G01N 2333/8146; G01N 2400/40; G01N 2800/52

(57) ABSTRACT

Methods for determining the presence, severity, and/or predisposition of Idiopathic Pulmonary Fibrosis (IPF) in an individual are disclosed. The methods utilize at least one diagnostic marker of a dynamic process of extracellular matrix synthesis and/or extracellular matrix degradation from said sample. The at least one diagnostic marker may be selected from the group consisting of tissue metallopeptidase inhibitor 1 (TIMP1), hyaluronan (HA), and/or Procollagen Type III N-terminal propeptide (PIIINP).

### CROSS REFERENCE TO RELATED APPLICATIONS

This application claims priority or the benefit under 35 U.S.C. § 119 of U.S. provisional patent application No. 63/363,282 filed on 20 Apr. 2022, the contents of which are fully incorporated herein by reference.

### SEQUENCE LISTING

The sequences referred to herein are listed in the Sequence Listing submitted as an ASCII text file entitled “biomarker_ST25a.xml”—5 KB and was created on 19 Apr. 2023 herein incorporated by reference in its entirety.

### BACKGROUND

Idiopathic pulmonary fibrosis (IPF) is a life-threatening fibrotic lung disease of unknown etiology that affects up to 185,000 individuals in the United States. There are no effective therapies for IPF, leading to significant morbidity and mortality. The pathogenesis of IPF is not fully understood. Although IPF was initially thought to result from generalized pulmonary inflammation leading to fibrosis, the current paradigm has shifted towards alveolar epithelial cell dysfunction and disordered fibroproliferation.

Although the median survival is 2-3 years, there is a wide spectrum of disease courses that can be generalized into three categories: stability or very slow decline, rapid deterioration, and periods of stability interspersed with periods of decline. Currently, it is difficult for clinicians to predict the disease course for an individual patient, as there are no accepted surrogates of these clinical courses.

Biomarkers act as surrogates for clinically meaningful outcomes and may or may not reflect the pathogenesis underlying a disease. Examples of clinical utility include diagnosis, the prediction of disease progression or regression, and prognostication of mortality. A biomarker should be easily acquired, reliably measured, and available for serial monitoring. Ideally, it would also provide an advantage of currently used clinical measures in ease, timeframe, and/or expense.

However, no reliable blood-based biomarkers have been identified as individual biomarkers or combination biomarkers (blood-based, imaging, or patient clinical data) that are effective at aiding in the diagnosis and progression of IPF.

Therefore, there is a need in the art for biomarkers and assays for IPF. It is to such biomarkers, as well as compositions/devices/assays containing reagents for measuring said biomarkers, along with methods of using same, that the present disclosure is directed.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 graphically depicts a receiver operator curve (ROC) illustrating the predictive value of the biomarker tissue metallopeptidase inhibitor 1 (TIMP1) alone to identify IPF, in accordance with the present disclosure.

FIG. 2 depicts an exemplary block diagram of a computer system 1100.

FIG. 3 depicts an exemplary flow chart of a method 1200.

FIG. 4 depicts an exemplary flow chart of a method 1300.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Before explaining at least one embodiment of the present disclosure in detail by way of exemplary language and results, it is to be understood that the present disclosure is not limited in its application to the details of construction and the arrangement of the components set forth in the following description. The present disclosure is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, devices, kits, and/or methods disclosed herein can be made and executed without undue experimentation in light of the present disclosure. While the compositions, devices, kits, and/or methods have been described in terms of particular embodiments, it will be apparent to those of skill in the art that variations may be applied to the compositions, devices, kits, and/or methods and in the steps or in the sequence of steps of the methods described herein without departing from the concept, spirit, and scope of the present disclosure. All such similar substitutions and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and, unless explicitly stated otherwise, is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” Is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

The term “antibody” is used herein in the broadest sense and refers to, for example, intact monoclonal antibodies and polyclonal antibodies, multi-specific antibodies (e.g., bispecific antibodies), antibody fragments and conjugates thereof that exhibit the desired biological activity of analyte binding (such as, but not limited to, Fab, Fab′, F(ab′)2, Fv, scFv, Fd, diabodies, single-chain antibodies, and other antibody fragments and conjugates thereof that retain at least a portion of the variable region of an intact antibody), antibody substitute proteins or peptides (i.e., engineered binding proteins/peptides), and combinations or derivatives thereof. The antibody can be of any type or class (e.g., IgG, IgE, IgM, IgD, and IgA) or sub-class (e.g., IgG1, IgG2, IgG3, IgG4, IgA1, and IgA2).

Biomarker: The term “biomarker” or “biological marker” is used herein, consistent with its use in the art, to refer to an entity whose presence, level, or form, correlates with a particular biological event or state of interest, so that it is considered to be a “marker” of that event or state. To give but a few examples, in some embodiments, a biomarker may be or include a marker for a particular disease state, or for likelihood that a particular disease, disorder or condition may develop, occur, or reoccur. In some embodiments, a biomarker may be or include a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. In some embodiments, a biomarker is a possible biomarker of the relevant biological event or state of interest. A biomarker may be an entity of any chemical class. For example, in some embodiments, a biomarker may be or include a nucleic acid, a polypeptide, a small molecule, or a combination thereof. In some embodiments, a biomarker is a cell surface marker. In some embodiments, a biomarker is intracellular. In some embodiments, a biomarker is found in a particular tissue (e.g., lung tissue). In some embodiments, a biomarker is found outside of cells (e.g., is secreted or is otherwise generated or present outside of cells, e.g., in a body fluid such as blood, urine, tears, saliva, cerebrospinal fluid, etc.

As described herein, in some embodiments, a biomarker is an IPF Biomarker. An “IPF Biomarker” as used herein refers to a biological marker for idiopathic pulmonary fibrosis (IPF). In some embodiments, one or more one or more IPF Biomarkers include tissue metallopeptidase inhibitor 1 (TIMP1), hyaluronan (HA), and Procollagen Type III N-terminal propeptide (PIIINP), or a combination thereof. In embodiments, an IPF Biomarker includes a gene product associated with the specific recited biomarker. For example, depending on context, “TIMP1” refers to a nucleotide encoding TIMP1 or a characteristic or functional fragment thereof, as well as a TIMP1 protein or a characteristic or functional fragment thereof.

Characteristic fragment: The term “characteristic fragment” refers to a fragment of a biomarker (e.g., IPF Biomarker) that is sufficient to identify the biomarker from which the fragment was derived. For example, in some embodiments, a “characteristic fragment” of a biomarker is one that contains an amino acid sequence, or a collection of amino acid sequences, that together allow for the biomarker from which the fragment was derived to be distinguished from other possible biomarkers, proteins, or polypeptides. In some embodiments, a characteristic fragment includes at least 10, at least 20, at least 30, at least 40, or at least 50 amino acids. In embodiments, a characteristic fragment refers to a fragment of a biomarker that has at least 90%, at least 95%, at least 99% sequence identity to the biomarker from which the characteristic fragment was derived.

Gene product or expression product: As used herein, the term “gene product” generally refers to an RNA transcribed from the gene (pre- and/or post-processing) or a polypeptide (pre- and/or post-modification) encoded by an RNA transcribed from the gene.

Hybridization: The term “hybridization” refers to the physical property of single-stranded nucleic acid molecules (e.g., DNA or RNA) to anneal to complementary nucleic acid molecules. Hybridization can typically be assessed in a variety of contexts—including where interacting nucleic acid molecules are studied in isolation or in the context of more complex systems (e.g., while covalently or otherwise associated with a carrier entity and/or in a biological system or cell). In some embodiments, hybridization can be detected by a hybridization technique, such as a technique selected from the group consisting of in situ hybridization (ISH), microarray, Northern blot, and Southern blot. In some embodiments, hybridization refers to 100% annealing between the single-stranded nucleic acid molecules and the complementary nucleic acid molecule. In some embodiments, annealing Is less than 100% (e.g., at least 95%, at least 90%, at least 85%, at least 80%, at least 75%, at least 70% of a single-stranded nucleic acid molecule anneals to a complementary nucleic acid molecule). Hybridization techniques, and methods for evaluating hybridization, are well known in the art. See, e.g., Sambrook, et al., 1989, Molecular Cloning: A Laboratory Manual, Second Edition, Cold Spring Harbor Press, Plainview, N.Y. Those skilled in the art understand how to estimate and adjust the stringency of hybridization conditions such that sequences having at least a desired level of complementary will stably hybridize, while those having lower complementary will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

Detection agent: The term “detection agent” as used herein refers to any element, molecule, functional group, compound, fragment or moiety that is detectable. In some embodiments, a detection agent is provided or utilized alone. In some embodiments, a detection agent is provided and/or utilized in association with (e.g., joined to) another agent. Examples of detection agents include, but are not limited to: various ligands, radionuclides (e.g., ³H, ¹⁴C, ¹⁸F, ¹⁹F, ³²P, ³⁵S, ¹³⁵I ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes, chemiluminescent agents (such as, for example, acridinium esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes, colorimetric labels (such as, for example, dyes, colloidal gold, and the like), biotin, digoxigenin, haptens, and proteins for which antisera or monoclonal antibodies are available.

Diagnostic test: As used herein, “diagnostic test” is a step or series of steps that is or has been performed to attain information that is useful in determining whether a patient has a disease, disorder or condition and/or in classifying a disease, disorder or condition into a phenotypic category or any category having significance with regard to prognosis of a disease, disorder or condition, or likely response to treatment (either treatment in general or any particular treatment) of a disease, disorder or condition. Similarly, “diagnosis” refers to providing any type of diagnostic information, including, but not limited to, whether a subject is likely to have or develop a disease, disorder or condition, state, staging or characteristic of a disease, disorder or condition as manifested in the subject, Information related to the nature or classification of a tumor, information related to prognosis and/or information useful in selecting an appropriate treatment or additional diagnostic testing. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc. Selection of additional diagnostic testing may include more specific testing for a given disease, disorder, or condition.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrase “associated with” includes both direct association of two moieties to one another as well as indirect association of two moieties to one another. Non-limiting examples of associations include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety.

The term “biological fluid sample” as used herein will be understood to include any liquid test sample that may be obtained from a patient and utilized in accordance with the present disclosure. Examples of biological fluid samples that may be utilized include, but are not limited to, whole blood or any portion thereof (i.e., plasma or serum), saliva, sputum, mucus, nasal, nasopharyngeal, anterior nasal, oropharyngeal, tracheal, bronchoalveolar, cerebrospinal fluid (CSF), intestinal fluid, intraperitoneal fluid, cystic fluid, sweat, interstitial fluid, tears, combinations thereof, and the like.

As used herein, the term “volume” as it relates to the liquid test samples utilized in accordance with the present disclosure typically refers to a volume of liquid test sample in a range of from about 0.1 μl to about 100 μl, or a range of from about 1 μl to about 75 μl, or a range of from about 2 μl to about 60 μl, or a value less than or equal to about 50 μl, or the like.

The term “patient” as utilized herein Includes human and veterinary subjects. In certain non-limiting embodiments, a patient is a mammal. In certain other non-limiting embodiments, the patient is a human. The term “mammal” for purposes of diagnosis/treatment refers to any animal classified as a mammal, including human, domestic and farm animals, nonhuman primates, and zoo, sports, or pet animals, such as dogs, horses, cats, cows, etc.

A “health care provider” or “health care decision maker” includes any individual authorized to diagnose or treat a patient, or to assist in the diagnosis or treatment of a patient. In the context of identifying useful new drugs to treat lung disease, a health care provider can be an individual who is not authorized to diagnose or treat a patient, or to assist in the diagnosis or treatment of a patient.

“Point of care testing” refers to real time diagnostic testing that can be done in a rapid time frame so that the resulting test is performed faster than comparable tests that do not employ this system. Point of care testing can be performed rapidly and on site, such as in a doctor's office, at a bedside, in a stat laboratory, emergency room, or other such locales, particularly where rapid and accurate results are required. The patient can be present, but such presence is not required. Point of care includes, but is not limited to: emergency rooms, operating rooms, hospital laboratories and other clinical laboratories, doctor's offices, in the field, or in any situation in which a rapid and accurate result is desired.

The term “specific binding partner,” as used in particular (but not by way of limitation) herein in the term “target analyte-specific binding partner,” will be understood to refer to any molecule capable of specifically associating with the target analyte. For example, but not by way of limitation, the binding partner may be an antibody, a receptor, a ligand, aptamers, molecular imprinted polymers (i.e., inorganic matrices), combinations or derivatives thereof, as well as any other molecules capable of specific binding to the target analyte.

The term “immunoassay” as utilized herein refers to an assay to determine the presence of a diagnostic biomarker in a biological sample by reacting the sample with an antibody (or fragment thereof) that specifically binds to the diagnostic biomarker, wherein the reaction is carried out for a time and under conditions that allow for the formation of an immunocomplex between the antibody (or fragment thereof) and the diagnostic biomarker. The quantitative determination of such an immunocomplex is then performed.

In certain particular (but non-limiting) embodiments, the immunoassays may detect an immobilized complex between a serum marker and a serum marker-binding antibody using a second antibody that is labeled and binds to the first antibody. Alternatively, the first version features a sandwich format in which the second antibody also binds a serum marker. In the sandwich immunoassay procedures, a serum marker-binding antibody can be a capture antibody attached to an insoluble material and the second antibody can be a labeling antibody. The above-described sandwich immunoassay procedures can be used with the antibodies described hereinafter.

In other particular (but non-limiting) embodiments, the immunoassays may detect a complex between a serum marker and a serum marker-binding antibody using a detection molecule (i.e., second reagent) that is capable of binding to the serum marker-binding antibody and also capable of being detected when bound to the immunocomplex. For example (but not by way of limitation), the second reagent may include a label attached to a receptor, a ligand, or even another copy of the serum marker.

Turning now to particular non-limiting embodiments of the present disclosure, embodiments of the present disclosure include individual biomarkers for IPF, as well as compositions/devices/assays containing same, methods of producing and using same, kits, and diagnostic tests related thereto. The identification of one or more biomarkers for IPF is attractive and advantageous for several reasons. First, the currently used diagnostic criteria for IPF rely on radiographic imaging and/or surgical lung biopsies interpreted by physicians with expertise in interstitial lung diseases. This expertise is often found only at tertiary care centers, which may be geographically distant from patients and their primary physicians. The identification of one or more serum biomarkers that are diagnostic for IPF will be helpful for both clinicians and patients, particularly in cases where a surgical lung biopsy cannot be obtained or access to dedicated interstitial lung disease physicians is limited. Secondly, the discovery of peripheral blood biomarker(s) that reflect disease activity will allow for serial monitoring as well as provide an objective marker(s) to assess treatment efficacy. Lastly, an IPF biomarker that provides prognostic information about disease course and/or mortality will be valuable for both clinical care and research study design. The present disclosure focuses on (but is not limited to) biomarkers that are present in peripheral blood, as they are easy to obtain, can be measured longitudinally, and have the greatest likelihood of achieving clinical utility. Prior to the present disclosure, there were no validated biomarkers that were routinely used in the clinical care of patients with IPF.

Certain non-limiting embodiments of the present disclosure are directed to a method of determining the presence, severity, and/or predisposition of Idiopathic Pulmonary Fibrosis (IPF) in an individual using a single biomarker. The method includes the steps of: (a) obtaining a biological fluid sample from an individual; (b) incubating the biological fluid sample with an antibody that specifically binds to tissue metallopeptidase inhibitor 1 (TIMP1) under conditions that allow for formation of an antibody-TIMP1 immunocomplex; (c) measuring an amount of antibody-TIMP1 immunocomplex formed to obtained a measured value for TIMP1 in the sample; and (d) using a mathematical algorithm to obtain an IPF score based on the measured value of TIMP1 in the sample.

Any biological fluid sample known in the art or otherwise contemplated herein that may contain TIMP1 that is detectable by an anti-TIMP1 antibody and that is indicative of IPF may be utilized in accordance with the present disclosure. Examples of biological fluid samples that may be utilized include, but are not limited to, blood, serum, plasma, saliva, sputum, mucus, nasal, nasopharyngeal, anterior nasal, oropharyngeal, tracheal, bronchoalveolar, combinations thereof, and the like.

Anti-TIMP1 antibodies are well known in the art, are widely commercially available, and have been vastly studied. For example (but not by way of limitation), a few commercial sources of ant-TIMP1 monoclonal and/or polyclonal antibodies include Abcam (Cambridge, UK); Biolegend, Inc. (San Jose, CA); Bio-Rad Laboratories, Inc. (Hercules, CA); Cell Signaling Technology, Inc. (Danvers, MA); Millipore Sigma (Burlington, MA); Santa Cruz Biotechnology, Inc. (Dallas, TX); Sino Biological US Inc. (Wayne, PA); Thermo Fisher Scientific (Waltham, MA); and many others. However, this list is not inclusive, and there are many additional commercial sources of anti-TIMP1 antibodies that can be utilized in accordance with the present disclosure. Thus, a person having ordinary skill in the art will clearly and unambiguously be able to identify and select a variety of anti-TIMP1 antibodies that can be utilized in accordance with the present disclosure, and as such, no further description of the anti-TIMP1 antibodies or the characteristics thereof is deemed necessary.

Non-limiting examples of TIMP1 immunoassays, reagents utilized therein, and algorithms that may be utilized in accordance with the present disclosure are described in detail in U.S. Pat. No. 7,141,380, issued Nov. 28, 2006; and U.S. Pat. No. 7,668,661, issued Feb. 23, 2010. The entire contents of each of the above-referenced patents are hereby expressly incorporated herein by reference.

In certain particular (but non-limiting) embodiments, the IPF score is used to support, predict, or substitute the histological score of a lung biopsy.

In certain particular (but non-limiting) embodiments, the mathematical algorithm is a discriminant function algorithm, such as (but not limited to) a linear discriminant function algorithm.

In certain particular (but non-limiting) embodiments, the IPF score is at least one factor to determine a treatment strategy for the individual.

In certain particular (but non-limiting) embodiments, the IPF score is at least one factor used to monitor the efficacy of an implemented treatment strategy for the individual.

In certain particular (but non-limiting) embodiments, the IPF score is at least one factor used to determine whether the individual should obtain a lung biopsy.

In certain particular (but non-limiting) embodiments, the IPF score is at least one factor used to evaluate the degree of IPF in the individual.

Certain non-limiting embodiments of the present disclosure are directed to a method of determining the presence, severity, and/or predisposition of Idiopathic Pulmonary Fibrosis (IPF) in an individual that utilizes two biomarkers. This method includes the steps of: (a) obtaining a biological fluid sample from an individual or patient; (b) selecting at least two diagnostic markers of a dynamic process of extracellular matrix synthesis and/or extracellular matrix degradation from said sample, wherein the at least two diagnostic markers are selected from the group consisting of tissue metallopeptidase inhibitor 1 (TIMP1), hyaluronan (HA), and Procollagen Type III N-terminal propeptide (PIIINP); (c) measuring the amount of each of the at least two diagnostic markers in the sample to obtain a measured value for each of the at least two diagnostic markers; and (d) combining the measured values of the at least two diagnostic markers using a mathematical algorithm to obtain an IPF score.

In certain particular (but non-limiting) embodiments, the at least two diagnostic markers are TIMP1 and HA.

In certain particular (but non-limiting) embodiments, the at least two diagnostic markers are TIMP1 and PIIINP.

In certain particular (but non-limiting) embodiments, the at least two diagnostic markers include TIMP1, HA, and PIIINP, and wherein step (d) is further defined as combining the measured values of the three diagnostic markers using the mathematical algorithm to obtain the IPF score. In embodiments, the TIMP1 and PIIINP (or P3NP) is characterized as human.

HA binding proteins and/or anti-HA antibodies are well known in the art, are widely commercially available, and have been vastly studied. For example (but not by way of limitation), a few commercial sources of anti-HA monoclonal and/or polyclonal antibodies include Abbexa Ltd (Houston, TX); Bio-Rad Laboratories, Inc. (Hercules, CA); Biorbyt Ltd. (St. Louis, MO); Creative Diagnostics (Shirley, NY); GeneTex, Inc. (Irvine, CA); LifeSpan BioSciences (Seattle, WA); MyBioSource, Inc. (San Diego, CA); US Biological Life Sciences (Salem, MA); and many others. However, this list is not inclusive, and there are many additional commercial sources of anti-HA antibodies that can be utilized in accordance with the present disclosure. Thus, a person having ordinary skill in the art will clearly and unambiguously be able to identify and select a variety of anti-HA antibodies that can be utilized in accordance with the present disclosure, and as such, no further description of the anti-HA antibodies or the characteristics thereof is deemed necessary.

Anti-PIIINP antibodies are well known in the art, are widely commercially available, and have been vastly studied. For example (but not by way of limitation), a few commercial sources of anti-PIIINP monoclonal and/or polyclonal antibodies include Abbexa Ltd (Houston, TX); Abcam (Cambridge, UK); Abnova Corporation (Walnut, CA); Antibodies-Online Inc. (Limerick, PA); Cedarlane (Burlington, Ontario); Creative Diagnostics (Shirley, NY); Millipore Sigma (Burlington, MA); MyBioSource, Inc. (San Diego, CA); Sino Biological US Inc. (Wayne, PA); and many others. However, this list is not inclusive, and there are many additional commercial sources of anti-PIIINP antibodies that can be utilized in accordance with the present disclosure. Thus, a person having ordinary skill in the art will clearly and unambiguously be able to identify and select a variety of anti-PIIINP antibodies that can be utilized in accordance with the present disclosure, and as such, no further description of the anti-PIIINP antibodies or the characteristics thereof is deemed necessary.

Non-limiting examples of TIMP1, HA, and PIIINP immunoassays, reagents utilized therein (including antibodies utilized therein), and algorithms that may be utilized in accordance with the present disclosure are described in detail in U.S. Pat. No. 7,141,380, issued Nov. 28, 2006; U.S. Pat. No. 7,668,661, issued Feb. 23, 2010; and U.S. Pat. No. 7,541,149, issued Jun. 2, 2009. The entire contents of each of the above-referenced patents are hereby expressly Incorporated herein by reference. In embodiments, a suitable biomarker for use herein includes a polypeptide having the amino acid sequence shown in (UniProt Accession No. P01033) such as:

(SEQ ID NO: 1)
MAPFEPLASGILLLLWLIAPSRACTCVPPHPQTAFCNSDLVIRAKFVGTP
EVNQTTLYQRYEIKMTKMYKGFQALGDAADIRFVYTPAMESVCGYFHRSH
NRSEEFLIAGKLQDGILHITTCSFVAPWNSLSLAQRRGFTKTYTVGCEEC
TVFPCLSIPCKLQSGTHCLWTDQLLQGSEKGFQSRHLACLPREPGLCTWQ
SLRSQIA,

or fragments or variants thereof, e.g., variants having at least 95%, at least 97%, or at least 99% sequence identity to this biomarker sequence. In embodiments, a suitable biomarker for use herein includes a polypeptide having the amino acid sequence shown in (UniProt Accession Nos. P02461 or P02461.1) such as:

(SEQ ID NO: 2)
MMSFVQKGSWLLLALLHPTIILAQQEAVEGGCSHLGQSYADRDVWKPEPC
QICVCDSGSVLCDDIICDDQELDCPNPEIPFGECCAVCPQPPTAPTRPPN
GQGPQGPKGDPGPPGIPGRNGDPGIPGQPGSPGSPGPPGICESCPTGPQN
YSPQYDSYDVKSGVAVGGLAGYPGPAGPPGPPGPPGTSGHPGSPGSPGYQ
GPPGEPGQAGPSGPPGPPGAIGPSGPAGKDGESGRPGRPGERGLPGPPGI
KGPAGIPGFPGMKGHRGFDGRNGEKGETGAPGLKGENGLPGENGAPGPMG
PRGAPGERGRPGLPGAAGARGNDGARGSDGQPGPPGPPGTAGFPGSPGAK
GEVGPAGSPGSNGAPGQRGEPGPQGHAGAQGPPGPPGINGSPGGKGEMGP
AGIPGAPGLMGARGPPGPAGANGAPGLRGGAGEPGKNGAKGEPGPRGERG
EAGIPGVPGAKGEDGKDGSPGEPGANGLPGAAGERGAPGFRGPAGPNGIP
GEKGPAGERGAPGPAGPRGAAGEPGRDGVPGGPGMRGMPGSPGGPGSDGK
PGPPGSQGESGRPGPPGPSGPRGQPGVMGFPGPKGNDGAPGKNGERGGPG
GPGPQGPPGKNGETGPQGPPGPTGPGGDKGDTGPPGPQGLQGLPGTGGPP
GENGKPGEPGPKGDAGAPGAPGGKGDAGAPGERGPPGLAGAPGLRGGAGP
PGPEGGKGAAGPPGPPGAAGTPGLQGMPGERGGLGSPGPKGDKGEPGGPG
ADGVPGKDGPRGPTGPIGPPGPAGQPGDKGEGGAPGLPGIAGPRGSPGER
GETGPPGPAGFPGAPGQNGEPGGKGERGAPGEKGEGGPPGVAGPPGGSGP
AGPPGPQGVKGERGSPGGPGAAGFPGARGLPGPPGSNGNPGPPGPSGSPG
KDGPPGPAGNTGAPGSPGVSGPKGDAGQPGEKGSPGAQGPPGAPGPLGIA
GITGARGLAGPPGMPGPRGSPGPQGVKGESGKPGANGLSGERGPPGPQGL
PGLAGTAGEPGRDGNPGSDGLPGRDGSPGGKGDRGENGSPGAPGAPGHPG
PPGPVGPAGKSGDRGESGPAGPAGAPGPAGSRGAPGPQGPRGDKGETGER
GAAGIKGHRGFPGNPGAPGSPGPAGQQGAIGSPGPAGPRGPVGPSGPPGK
DGTSGHPGPIGPPGPRGNRGERGSEGSPGHPGQPGPPGPPGAPGPCCGGV
GAAAIAGIGGEKAGGFAPYYGDEPMDFKINTDEIMTSLKSVNGQIESLIS
PDGSRKNPARNCRDLKFCHPELKSGEYWVDPNQGCKLDAIKVFCNMETGE
TCISANPLNVPRKHWWTDSSAEKKHVWFGESMDGGFQFSYGNPELPEDVL
DVHLAFLRLLSSRASQNITYHCKNSIAYMDQASGNVKKALKLMGSNEGEF
KAEGNSKFTYTVLEDGCTKHTGEWSKTVFEYRTRKAVRLPIVDIAPYDIG
GPDQEFGVDVGPVCFL,

or fragments or variants thereof, e.g., variants having at least 95%, at least 97%, or at least 99% sequence identity to this biomarker. % As used herein, the term “sequence identity” refers to the percent identity of bases or amino acids determined by comparing a first polynucleotide or polypeptide to a second polynucleotide or polypeptide using algorithms having various weighting parameters. Sequence identity between two polypeptides or two polynucleotides can be determined using sequence alignment by various methods and computer programs (e.g., BLAST, FASTA, L-ALIGN, etc.), available through the worldwide web at sites including GENBANK (ncbi.nlm.nih.gov/genbank/) and EMBL-EBI (ebi.ac.uk.). Sequence identity between two polynucleotides or two polypeptide sequences is generally calculated using the standard default parameters of the various methods or computer programs.

In certain particular (but non-limiting) embodiments, the IPF score is used to support, predict, or substitute the histological score of a lung biopsy.

In certain particular (but non-limiting) embodiments, the mathematical algorithm is a discriminant function algorithm, such as (but not limited to) a linear discriminant function algorithm.

In certain particular (but non-limiting) embodiments, the IPF score is at least one factor to determine a treatment strategy for the individual.

In certain particular (but non-limiting) embodiments, the IPF score is at least one factor used to monitor the efficacy of an Implemented treatment strategy for the individual.

In certain particular (but non-limiting) embodiments, the IPF score is at least one factor used to determine whether the individual should obtain a lung biopsy.

In certain particular (but non-limiting) embodiments, the IPF score is at least one factor used to evaluate the degree of IPF in the individual.

The current reference standard to assess fibrosis in the lung is the lung biopsy. In a biopsy, tissue samples randomly taken out of the lung are cut into slices which are examined by an expert using a microscope.

There are numerous problems associated with lung biopsies, including the following sources of uncertainty: distribution of fibrosis in the lung (where there is clustered fibrosis, the needle might have hit regions of the lung not affected by fibrosis), failed sample preparation (e.g. not enough tissue material), and pathologist subjectivity. Furthermore, the fibrotic state of the lung is usually described using scores and there are many different, and possibly incompatible, scoring systems (e.g., CRP-scoring systems. For example, two independent pathologists may have to score the same biopsy samples for the same patient at two different time-points using two different scoring systems.

The present disclosure facilitates point of care or remote diagnoses of IPF and assists health care providers in monitoring the status or progress of IPF at two or more time points. Significantly, the present disclosure provides health care decision makers with an alternative to potentially inaccurate and risky lung biopsies.

The present disclosure employs computer-implementable algorithmic methods which utilize one or more IPF-related marker values. The predictive value of the present disclosure has been validated in clinical studies which monitored the status or progress of IPF. These clinical trials validated the present disclosure on a cross-sectional basis, in which analyses were conducted at discrete time points, and longitudinally, in which analyses were conducted at two or more time points.

Accordingly, the present disclosure can be used to: (a) measure the dynamic processes of extracellular matrix synthesis (fibrogenesis) and extracellular matrix degradation (fibrolysis); and (b) obtain results that reflect the degree of fibrosis and the dynamic changes occurring in lung tissue through prediction of an IPF histological score.

The present disclosure is especially useful in aiding in the diagnosis and treatment of patients for whom a lung biopsy would be very risky. Such patients may suffer from coagulopathy, may be averse to undergoing a biopsy, or may not have access to expert histopathology. In addition, the present disclosure can be used by health care decision makers to assess IPF. Further, the present disclosure is especially useful in cases where fibrosis may be unevenly distributed, and sampling errors pose a significant problem.

In one non-limiting embodiment, the present disclosure provides a method that aids in the diagnosis of the status or progress of IPF in a patient by determining at one or more time points a predictor value for each time point, wherein a comparison at one or more time points of the predictor value and a comparative data set is used by a health care decision maker to ascertain the status or progress of patient IPF, and wherein patient predictor values are calculated by inputting data for one or more blood markers (e.g., one or more plasma or serum markers), and optionally one or more supplementary markers, into a linear or nonlinear function algorithm derived by correlating reference IPF histopathological and blood markers (e.g., plasma or serum marker data).

A “comparative data set” can include any data reflecting any qualitative or quantitative indicia of histopathological conditions. In one non-limiting embodiment, the comparative data set can include one or more numerical values, or range of numerical values, associated with histopathological conditions. For example, a comparative data set may include various integer sets (e.g., the integers 0 through 5), wherein different groupings of those six integers correlate to different IPF disease states (e.g., 0.1 may correlate to a mild disease state, 2-3 correlate to a moderate disease state, and 4-5 may correlate to a severe disease state). Therefore, a comparative data set may correlate to an established lung biopsy scoring system (e.g., clinical-radiographic-physiologic (CRP) scoring system).

In a particular (but non-limiting) embodiment, blood markers are serum markers that are selected from one or more of the following: tissue metallopeptidase inhibitor 1 (TIMP1), N-terminal procollagen Ill propeptide (PIIINP), and Hyaluronan. Supplementary markers include, but are not limited to, patent weight, sex, age, and transaminase level.

In another non-limiting embodiment of the present disclosure, the linear or nonlinear function algorithm is derived by correlating reference IPF histopathological and blood marker (e.g., plasma and serum marker) data using either discriminant function analysis or nonparametric regression analysis. Reference IPF histopathological and blood marker data (e.g., plasma and serum marker data) can include data indicative of fibrogenesis or fibrolysis, elevated IPF serum markers, or other IPF clinical symptoms.

In one non-limiting embodiment, reference IPF histopathological and blood marker data (e.g., plasma and serum marker data) is based upon data relating to one or more subjects other than the diagnosed patient. In another non-limiting embodiment, reference IPF histopathological and blood marker data (e.g., plasma and serum marker data) is based upon data previously obtained from the diagnosed patient, and is optionally also based on data obtained from one or more other subjects.

In one non-limiting embodiment, a linear or nonlinear function algorithm is derived by correlating reference IPF histopathological and blood marker data (e.g., plasma and serum marker data) by: (a) compiling a data set including blood marker data (e.g., plasma or serum marker data) and histopathological data for a first group of subjects; (b) deriving a linear or nonlinear function algorithm from the compiled data set through application of an analytical methodology; (c) calculating validation biopsy score values for a second group of subjects by inputting data including blood marker data (e.g., plasma or serum marker data) values for the second group of subjects into the algorithm derived in step (b); and (d) comparing validation biopsy score values calculated in step (c) with IPF histopathological scores for the second group of subjects; and (e) if the validation biopsy scores determined in step (c) do not correlate within a clinically-acceptable tolerance level with IPF histopathological scores for the second group of subjects, performing the following operations (i)-(iii) until such tolerance is satisfied: (i) modifying the algorithm on a basis or bases including (1) revising the data set for the first group of subjects, and (2) revising or changing the analytical methodology (ii) calculating validation biopsy score values for the second group of subjects by inputting data including blood marker data (e.g., plasma or serum marker data) values for the second group of subjects into the modified algorithm (iii) assessing whether validation biopsy score values calculated using the modified algorithm correlate with lung histopathological scores for the second group of subjects within the clinically-acceptable tolerance level.

The analytical methodology may include statistical techniques including discriminant function analysis and nonparametric regression analysis, as well as techniques such as classification trees or neural networks.

In another non-limiting embodiment, the present disclosure provides a data structure stored in a computer-readable medium that may be read by a microprocessor and that includes at least one code that uniquely identifies a linear or nonlinear function algorithm derived in a manner described herein.

In another non-limiting embodiment, the present disclosure provides a diagnostic kit including: (a) a data structure stored in a computer-readable medium that may be read by a microprocessor and that includes at least one code that uniquely identifies a linear or nonlinear function algorithm derived in a manner described herein; and (b) one or more immunoassays that detect and determine patient serum marker values.

In another non-limiting embodiment, the present disclosure provides computer-implementable methods and systems for determining whether a composition is useful in the treatment of IPF including evaluating data useful in diagnosing the status or progress of IPF in a patient treated with the composition, wherein: (a) the diagnosis is made by a health care provider by determining algorithmically at one or more time points a predictor value for each time point; (b) a comparison at one or more time points of the predictor value and a comparative data set is used by a health care provider to ascertain the status or progress of patient IPF; and (c) patient predictor values are calculated by inputting data for one or more blood markers (e.g., plasma or serum markers) into a linear or nonlinear function algorithm derived by correlating reference IPF histopathological and blood marker data (e.g., plasma or serum marker data).

The aforementioned methods, systems, and kits of the present disclosure can also be used by health care providers to: (1) determine treatment regimens for patients that are predisposed to, or suffer from, IPF; and (2) design clinical programs useful in monitoring the status or progress of IPF in one or more patients.

“Discriminant function analysis” is a technique used to determine which variables discriminate between two or more naturally occurring mutually exclusive groups. The basic idea underlying discriminant function analysis is to determine whether groups differ with regard to a set of predictor variables which may or may not be independent of each other, and then to use those variables to predict group membership (e.g., of new cases).

Discriminant function analysis starts with an outcome variable that is categorical (two or more mutually exclusive levels). The model assumes that these levels can be discriminated by a set of predictor variables which, like ANOVA (analysis of variance), can be continuous or categorical and, like ANOVA, assumes that the underlying discriminant functions are linear. Discriminant analysis does not “partition variation.” It does look for canonical correlations among the set of predictor variables and uses these correlates to build eigenfunctions that explain percentages of the total variation of all predictor variables over all levels of the outcome variable.

The output of the analysis is a set of linear discriminant functions (eigenfunctions) that use combinations of the predictor variables to generate a “discriminant score” regardless of the level of the outcome variable. The percentage of total variation is presented for each function. In addition, for each eigenfunction, a set of Fisher Discriminant Functions are developed that produce a discriminant score based on combinations of the predictor variables within each level of the outcome variable.

Usually, several variables are included in a study in order to see which one(s) contribute to the discrimination between groups. In that case, a matrix of total variances and co-variances is generated. Similarly, a matrix of pooled within-group variances and co-variances may be generated. A comparison of those two matrices via multivariate F tests is made in order to determine whether or not there are any significant differences (with regard to all variables) between groups. This procedure is identical to multivariate analysis of variance or MANOVA. As in MANOVA, one could first perform the multivariate test, and, if statistically significant, proceed to see which of the variables have significantly different means across the groups.

For a set of observations containing one or more quantitative variables and a classification variable defining groups of observations, the discrimination procedure develops a discriminant criterion to classify each observation into one of the groups. In order to get an idea of how well a discriminant criterion “performs,” it is necessary to classify (a priori) different cases, that is, cases that were not used to estimate the discriminant criterion. Only the classification of new cases enables an assessment of the predictive validity of the discriminant criterion.

In order to validate the derived criterion, the classification can be applied to other data sets. The data set used to derive the discriminant criterion is called the training or calibration data set or patient training cohort. The data set used to validate the performance of the discriminant criteria is called the validation data set or validation cohort.

The discriminant criterion (function(s) or algorithm), determines a measure of generalized squared distance. These distances are based on the pooled co-variance matrix. Either Mahalanobis or Euclidean distance can be used to determine proximity. These distances can be used to identify groupings of the outcome levels and so determine a possible reduction of levels for the variable.

A “pooled co-variance matrix” is a numerical matrix formed by adding together the components of the covariance matrix for each subpopulation in an analysis.

A “predictor” is any variable that may be applied to a function to generate a dependent or response variable or a “predictor value.” In one non-limiting embodiment of the present disclosure, a predictor value may be a discriminant score determined through discriminant function analysis of two or more patient blood markers (e.g., plasma or serum markers). For example, a linear model specifies the (linear) relationship between a dependent (or response) variable Y, and a set of predictor variables, the X's, so that

$$Y = {b}_{0} + {b}_{1} {X}_{1} + {b}_{2} {X}_{2} + … + {b}_{k} {X}_{k}$$

In this equation b₀ is the regression coefficient for the intercept and the b, values are the regression coefficients (for variables 1 through k) computed from the data.

“Classification trees” are used to predict membership of cases or objects in the classes of a categorical dependent variable from their measurements on one or more predictor variables. Classification tree analysis is one of the main techniques used in so-called Data Mining. The goal of classification trees is to predict or explain responses on a categorical dependent variable, and as such, the available techniques have much in common with the techniques used in the more traditional methods of Discriminant Analysis, Cluster Analysis, Nonparametric Statistics, and Nonlinear Estimation.

The flexibility of classification trees makes them a very attractive analysis option, but this is not to say that their use is recommended to the exclusion of more traditional methods. Indeed, when the typically more stringent theoretical and distributional assumptions of more traditional methods are met, the traditional methods may be preferable. But as an exploratory technique, or as a technique of last resort when traditional methods fail, classification trees are, in the opinion of many researchers, unsurpassed. Classification trees are widely used in applied fields as diverse as medicine (diagnosis), computer science (data structures), botany (classification), and psychology (decision theory). Classification trees readily lend themselves to being displayed graphically, helping to make them easier to interpret than they would be if only a strict numerical interpretation were possible.

“Neural Networks” are analytic techniques modeled after the (hypothesized) processes of learning in the cognitive system and the neurological functions of the brain and capable of predicting new observations (on specific variables) from other observations (on the same or other variables) after executing a process of so-called learning from existing data. Neural Networks is one of the Data Mining techniques. The first step is to design a specific network architecture (that includes a specific number of “layers” each consisting of a certain number of “neurons”). The size and structure of the network needs to match the nature (e.g., the formal complexity) of the investigated phenomenon. Because the latter is obviously not known very well at this early stage, this task is not easy and often involves multiple “trials and errors.”

The neural network is then subjected to the process of “training.” In that phase, computer memory acts as neurons that apply an iterative process to the number of inputs (variables) to adjust the weights of the network in order to optimally predict the sample data on which the “training” Is performed. After the phase of learning from an existing data set, the new network is ready and it can then be used to generate predictions.

In one non-limiting embodiment of the present disclosure, neural networks can include memories of one or more personal or mainframe computers or computerized point of care device.

While the present disclosure will be described in the general context of computer-executable instructions of a computer program that runs on a personal computer, those skilled in the art will recognize that the present disclosure also may be implemented in combination with other program modules. Generally, program modules include routines, programs, components, and data structures that perform particular tasks or implement particular abstract data types. Moreover, those skilled in the art will appreciate that the present disclosure may be practiced with other computer system configurations, including hand-held devices, multi-processor systems, microprocessor-based or programmable consumer electronics, minicomputers, mainframe computers, and the like. The present disclosure may also be practiced in distributed computing environments where tasks are performed by remote processing devices that are linked through a communications network. In a distributed computing environment, program modules may be located in both local and remote memory storage devices.

A diagnostic system of the present disclosure may include a handheld device useful in point of care applications or may be a system that operates remotely from the point of patient care. In either case the system can include companion software programmed in any useful language to implement diagnostic methods of the present disclosure in accordance with algorithms or other analytical techniques described herein.

“Validation cohort marker score values” means a numerical score derived from the linear combination of the discriminant weights obtained from the training cohort and marker values for each patient in the validation cohort

“Patient diagnostic marker cut-off values” means the value of a marker or combination of markers at which a predetermined sensitivity or specificity is achieved.

“Negative Predictive Power” (“NPV”) refers to the probability of not having a disease given that a marker value (or set of marker values) is not elevated above a defined cutoff.

“Positive Predictive Value” (“PPV”) refers to the probability of having a disease given that a maker value (or set of marker values) is elevated above a defined cutoff

“Receiver Operator Characteristic Curve” (“ROC”) refers to a graphical representation of the functional relationship between the distribution of a marker's sensitivity and 1-specificity values in a cohort of diseased persons and in a cohort of non-diseased persons.

“Area Under the Curve” (“AUC”) is a number which represents the area under a Receiver Operator Characteristic curve. The closer this number is to one, the more the marker values discriminate between diseased and non-diseased cohorts

“McNemar Chi-square Test” (“The McNemar χ² test”) is a statistical test used to determine if two correlated proportions (proportions that share a common numerator but different denominators) are significantly different from each other.

A “nonparametric regression analysis” is a set of statistical techniques that allows the fitting of a line for bivariate data that makes little or no assumptions concerning the distribution of each variable or the error in estimation of each variable. Non-limiting examples include Theil estimators of location, Passing-Bablok regression, and Deming regression.

“Cut-off values” are numerical values of a marker (or set of markers) that define a specified sensitivity or specificity.

### Kits

Also provided by the present disclosure are kits including one or more anti-IPF Biomarker agents and instructions for use (e.g., treatment, prophylactic, or diagnostic use). In some embodiments, the kit is used for an in vitro diagnostic assay to diagnose IPF. In some embodiments, the one or more anti-IPF Biomarker agents include antibody agents. In some embodiments, one or more of the antibody agents are labeled with a detectable moiety. In some embodiments, the kit further includes a detection agent (e.g., one or more acridinium ester molecules). In some embodiments, one or more of the antibody agents are labeled with one or more of the acridinium ester molecules. In some embodiments, the kit further includes one or more secondary antibody agents that specifically bind to one or more of the anti-IPF Biomarker antibody agents.

In some embodiments, the one or more anti-IPF Biomarker agents include nucleic acid probes. In some embodiments, at least a portion of each nucleic acid probe hybridizes to one or more portions of a nucleotide that encodes an IPF Biomarker (e.g., tissue metallopeptidase inhibitor 1 (TIMP1), hyaluronan (HA), and Procollagen Type Ill N-terminal propeptide (PIIINP), or combinations thereof). Nucleotides that encode an IPF Biomarker can be DNA (e.g., cDNA) or RNA (e.g. mRNA). In some embodiments, the nucleic acid probes are labeled with one or more detection agents (e.g., wherein the detection agents indicate presence of nucleotides that encode an IPF Biomarker).

In some embodiments, the kit further includes one or more control samples. In some embodiments, the control samples include one or more IPF Biomarker standards. In some embodiments, an IPF Biomarker standard includes recombinant IPF Biomarker (e.g., tissue metallopeptidase inhibitor 1 (TIMP1), hyaluronan (HA), and Procollagen Type III N-terminal propeptide (PIIINP), or combinations thereof). In some embodiments, an IPF Biomarker standard includes synthetic IPF Biomarker (e.g., tissue metallopeptidase inhibitor 1 (TIMP1), hyaluronan (HA), and Procollagen Type III N-terminal propeptide (PIIINP), or combinations thereof) nucleic acids.

In addition to the above, a kit can include other ingredients, such as a solvent or buffer, a stabilizer or a preservative, and/or an agent for treating a condition or disorder described herein. Alternatively, other ingredients can be included in a kit, but in different compositions or containers than the anti-IPF Biomarker agents. In such embodiments, a kit can include instructions for admixing the anti-IPF Biomarker agents and the other ingredients, or for using the anti-IPF Biomarker together with the other ingredients.

In certain embodiments, kits for use in accordance with the present disclosure may include, a reference or control sample(s), instructions for processing samples, performing tests on samples, instructions for interpreting the results, buffers and/or other reagents necessary for performing tests.

The present disclosure also provides that recognition that certain single IPF Biomarkers can be helpful for detecting and/or diagnosing IPF. The present disclosure further provides the insight that particular combinations of IPF Biomarkers are especially useful for detecting and/or diagnosing IPF. Thus, methods, compositions, and kits described herein can be used for assays to assess the risk of IPF, assess whether a subject should undergo further pulmonary tests, and/or diagnose IPF based on detection or measurement of IPF Biomarkers in a sample, e.g., a biological sample obtained from a subject.

Methods and kits provided herein are able to detect IPF in a sample with a sensitivity and a specificity that renders the outcome of the test reliable enough to be medically actionable. Methods and kits described herein for detection and/or diagnosis of IPF in a subject detects IPF with a sensitivity greater than 75%, greater than 80%, greater than 85%, greater than 90%, greater than 95%, greater than 96%, greater than 97%, greater than 98%, greater than 99%, or about 100%. In some embodiments, methods and kits provided herein can detect IPF with a sensitivity that is between about 70%-100%, between about 80%-100%, or between about 90-100%. In some embodiments, methods and kits provided herein can detect IPF with a sensitivity and a specificity that is between about 50%-100%, between about 60%-100%, between about 70%-100%, between about 80%-100%, or between about 90-100%.

### Compositions

Also provided herein are compositions. In some embodiments, a composition includes one or more IPF Biomarkers and one or more anti-IPF Biomarker agents. In some embodiments, one or more IPF Biomarkers include tissue metallopeptidase inhibitor 1 (TIMP1), hyaluronan (HA), and Procollagen Type Ill N-terminal propeptide (PIIINP), or combinations thereof, and one or more anti-IPF Biomarker agents include an anti-TIMP1 agent, an anti-HA agent, an anti-PIIINP agent, or a combination thereof.

In some embodiments, a composition includes a combination (e.g., one or more, two or more, or three of IPF Biomarkers and a corresponding combination of anti-IPF Biomarker agents. In embodiments, the one or more, two or more, or three of IPF Biomarkers are present in an amount sufficient to demonstrate the presence of IPF in a patient.

In some embodiments, a composition includes two or more IPF Biomarkers and two or more anti-IPF Biomarker agents. In some embodiments, two or more IPF Biomarkers include tissue metallopeptidase inhibitor 1 (TIMP1), hyaluronan (HA), and Procollagen Type III N-terminal propeptide (PIIINP), or a combinations thereof, and two or more anti-IPF Biomarker agents include an anti-TIMPlagent, an anti-HA agent, an anti-PIIINP agent, or a combination thereof.

In some embodiments, a composition includes three IPF Biomarkers and three or more anti-IPF Biomarker agents. In some embodiments, three IPF Biomarkers include tissue metallopeptidase inhibitor 1 (TIMP1), hyaluronan (HA), and Procollagen Type Ill N-terminal propeptide (PIIINP), or a combinations thereof. In some embodiments, three or more anti-IPF Biomarker agents include an anti-TIMP1 agent, an anti-HA agent, an anti-PIIINP agent, or a combination thereof.

### Computer Systems

Methods described herein can be implemented in a computer system having a processor that executes specific instructions in a computer program. In some embodiments, a computer system may be arranged to output an IPF Biomarker score based on receiving an IPF Biomarker profile and/or a level of two or more IPF Biomarkers. Particularly, a computer program may include instructions for the system to select appropriate next steps, including additional medication, a treatment, and/or additional testing for a subject.

In some embodiments, the computer program may be configured such that the computer system can identify a subject for further testing (e.g., pulmonary tests), identify a subject as being at risk or having IPF, and/or identify a subject to receive medication based on received data (e.g., an IPF Biomarker profile) and use the data to calculate an IPF Biomarker score. A system may be able to rank-order identified next steps based on an IPF Biomarker profile with demographic factors and/or Imaging-based biomarkers. A system may be able to adjust the rank ordering based on, e.g., a clinical response of a subject or of a family member of the subject who has or is suspected of having IPF.

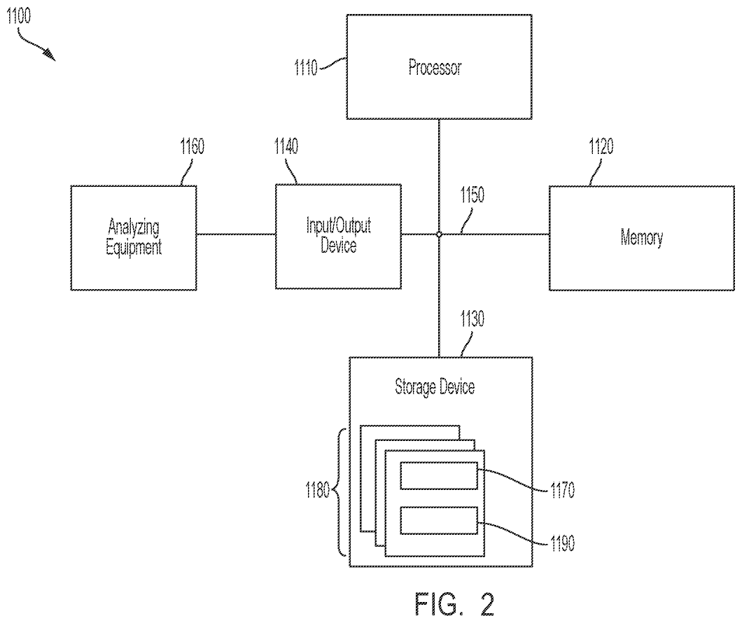

FIG. 2 is a block diagram of a computer system 1100 that can be used in the operations described above, according to one embodiment. The system 1100 includes a processor 1110, a memory 1120, a storage device 1130 and an input/output device 1140. Each of the components 1110, 1120, 1130 and 1140 are interconnected using a system bus 1150. The system may include analyzing equipment 1160 for determining a level of one or more biomarkers of the present disclosure in a sample.

In embodiments, the processor 1110 is capable of processing instructions for execution within the system 1100. In one embodiment, the processor 1110 is a single-threaded processor. In another embodiment, the processor 1110 is a multi-threaded processor. The processor 1110 is capable of processing instructions stored in the memory 1120 or on the storage device 1130, including for receiving or sending information through the input/output device 1140.

In embodiments, the memory 1120 stores information within the system 1100. In one embodiment, the memory 1120 is a computer-readable medium. In one embodiment, the memory 1120 is a volatile memory unit. In another embodiment, the memory 1120 is a non-volatile memory unit.

The storage device 1130 is capable of providing mass storage for the system 1100. In one embodiment, the storage device 1130 is a computer-readable medium.

The input/output device 1140 provides input/output operations for the system 1100. In one embodiment, the input/output device 1140 includes a keyboard and/or pointing device. In one embodiment, the input/output device 1140 includes a display unit for displaying graphical user interfaces.

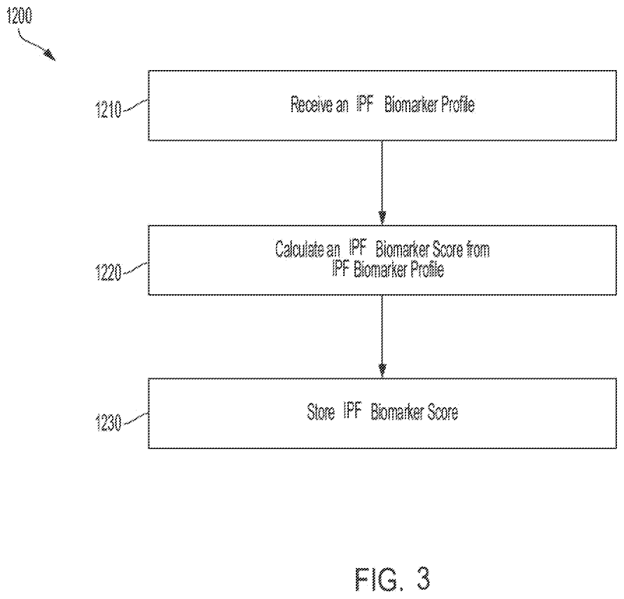

The system 1100 can be used to build a database. FIG. 3 shows a flow chart of a method 1200 for building a database for use in identifying a subject for further testing (e.g., IPF tests), identifying a subject as being at risk or having IPF, and/or identifying a subject to receive medication. Preferably, a method 1200 is performed in a system 1100. For example, a computer program product can include instructions that cause a processor 1110 to perform the steps of a method 1200 or a method 1300.

Referring now to FIG. 3, method 1200 includes the following steps. Receiving, In step 1210, a subject's IPF Biomarker Profile (e.g., levels of one or more IPF Biomarkers in a sample). A computer program in the system 1100 may include instructions for presenting a suitable graphical user interface on input/output device 1140, and the graphical user interface may prompt the user to enter the levels 1170 using the input/output device 1140, such as a keyboard. Calculating, in step 1220, an IPF Biomarker score from an IPF Biomarker profile. As described herein, calculating, in step 1220, an IPF Biomarker score from (i) an IPF Biomarker profile and (ii) demographic factors and/or image-based biomarkers. Storing, in step 1230, an IPF Biomarker score. The system 1100 may store an IPF Biomarker score in the storage device 1130. Additionally or alternatively, a system 1100 may provide a readout including an IPF Biomarker score. A readout may also include proposed next steps for a subject and/or a confidence level associated with an IPF Biomarker score.

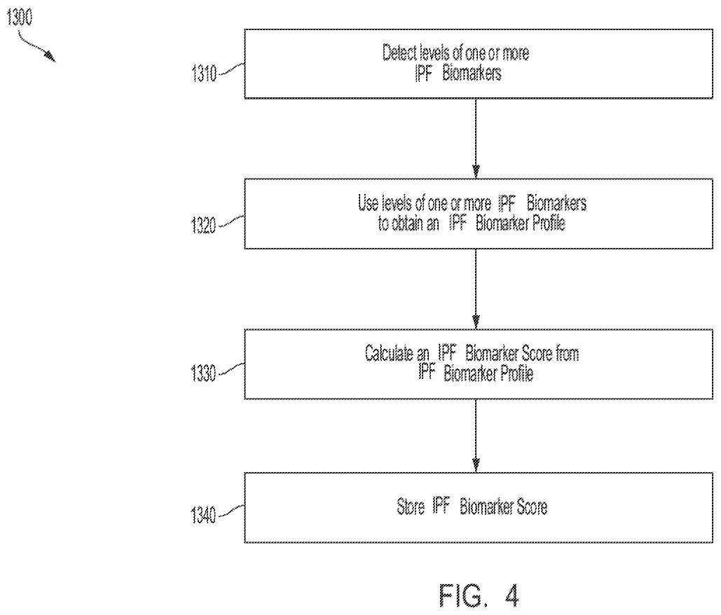

Referring now to FIG. 4, method 1300 includes the following steps. Detecting, in step 1310, levels of one or more IPF Biomarkers in a sample, e.g., from a subject. Using, in step 1320, levels of one or more IPF Biomarkers to obtain an IPF Biomarker Profile. Calculating, in step 1330, an IPF Biomarker score from an IPF Biomarker profile. As described herein, calculating, in step 1330, an IPF Biomarker score from (i) an IPF Biomarker profile and (ii) demographic factors and/or image-based biomarkers. Storing, in step 1340, an IPF Biomarker score. The system 1100 may store an IPF Biomarker score in the storage device 1130. Additionally or alternatively, a system 1100 may provide a readout including an IPF Biomarker score. A readout may also include proposed next steps for a subject and/or a confidence level associated with an IPF Biomarker score.

Additionally, non-transitory computer readable media containing executable instructions that when executed cause a processor to perform operations including a method as provided herein are provided. For example, a non-transitory computer readable medium containing executable instructions that when executed cause a processor to perform operations including a method of 1200 or 1300 described above. In embodiments, a non-transitory computer readable medium includes a hard drive, external hard drive, discs, CDs, DVDs, and the like that stores data. In embodiments, software disposed within a physical medium is suitable for use herein.

In some embodiments, a non-transitory computer readable media containing executable instructions that when executed cause a processor to perform operations including a method of determining the presence, severity, and/or predisposition of Idiopathic Pulmonary Fibrosis (IPF) in an individual, the method including the steps of: (a) incubating a biological fluid sample with an antibody that specifically binds to tissue metallopeptidase inhibitor 1 (TIMP1) under conditions that allow for formation of an antibody-TIMP1 immunocomplex; (b) measuring an amount of antibody-TIMP1 immunocomplex formed to obtained a measured value for TIMP1 in the sample; and (c) using a mathematical algorithm to obtain an IPF score based on the measured value of TIMP1 in the sample.

### Exemplary Numbered Embodiments

Embodiment 1. A method of determining the presence, severity, and/or predisposition of Idiopathic Pulmonary Fibrosis (IPF) in an individual, the method including the steps of: (a) obtaining a biological fluid sample from an individual; (b) incubating the biological fluid sample with an antibody that specifically binds to tissue metallopeptidase inhibitor 1 (TIMP1) under conditions that allow for formation of an antibody-TIMP1 immunocomplex; (c) measuring an amount of antibody-TIMP1 immunocomplex formed to obtained a measured value for TIMP1 in the sample; and (d) using a mathematical algorithm to obtain an IPF score based on the measured value of TIMP1 in the sample.

Embodiment 2. The method of embodiment 1, wherein the biological fluid sample is selected from the group consisting of blood, serum, plasma, saliva, sputum, mucus, nasal, nasopharyngeal, anterior nasal, oropharyngeal, tracheal, bronchoalveolar, and combinations thereof.

Embodiment 3. The method of embodiment 1, wherein the IPF score is used to support, predict, or substitute the histological score of a lung biopsy.

Embodiment 4. The method of embodiment 1, wherein the mathematical algorithm is a discriminant function algorithm.

Embodiment 5. The method of embodiment 1, wherein the discriminant function algorithm is a linear discriminant function algorithm.

Embodiment 6. The method of embodiment 1, wherein the IPF score is at least one factor to determine a treatment strategy for the individual.

Embodiment 7. The method of embodiment 1, wherein the IPF score is at least one factor used to monitor the efficacy of an implemented treatment strategy for the individual.

Embodiment 8. The method of embodiment 1, wherein the IPF score is at least one factor used to determine whether the individual should obtain a lung biopsy.

Embodiment 9. The method of embodiment 1, wherein the IPF score is at least one factor used to evaluate the degree of IPF in the individual.

Embodiment 10. A method of determining the presence, severity, and/or predisposition of Idiopathic Pulmonary Fibrosis (IPF) in an individual, the method including the steps of: (a) obtaining a biological fluid sample from an individual; (b) selecting at least two diagnostic markers of a dynamic process of extracellular matrix synthesis and/or extracellular matrix degradation from said sample, wherein the at least two diagnostic markers are selected from the group consisting of tissue metallopeptidase inhibitor 1 (TIMP1), hyaluronan (HA), and Procollagen Type III N-terminal propeptide (PIIINP); (c) measuring the amount of each of the at least two diagnostic markers in the sample to obtain a measured value for each of the at least two diagnostic markers; and (d) combining the measured values of the at least two diagnostic markers using a mathematical algorithm to obtain an IPF score.

Embodiment 11. The method of embodiment 10, wherein the at least two diagnostic markers are TIMP1 and HA.

Embodiment 12. The method according to embodiment 10, wherein the at least two diagnostic markers are TIMP1 and PIIINP.

Embodiment 13: The method according to embodiment 10, wherein the at least two diagnostic markers include TIMP1, HA, and PIIINP, and wherein step (d) is further defined as combining the measured values of the three diagnostic markers using the mathematical algorithm to obtain the IPF score.

Embodiment 14. The method according to embodiment 10, wherein the IPF score is used to support, predict, or substitute the histological score of a lung biopsy.

Embodiment 15. The method according to embodiment 10, wherein the mathematical algorithm is a discriminant function algorithm.

Embodiment 16. The method according to embodiment 15, wherein the discriminant function algorithm is a linear discriminant function algorithm.

Embodiment 17. The method according to embodiment 16, wherein the IPF score is at least one factor to determine a treatment strategy for the individual.

Embodiment 18. The method according to embodiment 10, wherein the IPF score is at least one factor used to monitor the efficacy of an implemented treatment strategy for the individual.

Embodiment 19. The method according to embodiment 10, wherein the IPF score is at least one factor used to determine whether the individual should obtain a lung biopsy.

Embodiment 20. The method according to embodiment 10, wherein the IPF score is at least one factor used to evaluate the degree of IPF in the individual.

Embodiment 21. A non-transitory computer readable medium containing executable instructions that when executed cause a processor to perform operations including the method of any one of embodiments 1-20.

Embodiment 22. A composition including:
(a) one or more IPF Biomarkers, wherein the one or more IPF Biomarkers include: tissue metallopeptidase inhibitor 1 (TIMP1), hyaluronan (HA), and Procollagen Type III N-terminal propeptide (PIIINP), or a combination thereof; and (b) one or more anti-IPF Biomarker agents, wherein the one or more anti-IPF Biomarker agents include: an anti-TIMP1 agent, an anti-HA agent, an anti-PIIINP agent, or a combination thereof. In embodiments, the one or more anti-IPF Biomarker agents are man-made or synthetic.

Embodiment 23: A kit for detecting IPF, said kit including: (a) one or more anti-IPF Biomarker agents, wherein the one or more anti-IPF Biomarker agents include:
(i) an anti-TIMPlagent,
(ii) an anti-HA agent,
(iii) an anti-PIIINP agent,
(iv) a combination thereof; and
(b) instructions for use.

Embodiment 24: A kit for detecting IPF, said kit including: (a) one or more anti-IPF Biomarker agents, wherein the one or more anti-IPF Biomarker agents include (i) an anti-TIMP1 agent and an anti-HA agent, (ii) an anti-PIIINP agent and an anti-TIMP1 agent, or (iii) an anti-HA agent, an anti-PIIINP agent, and an anti-TIMP1 agent; and (b) instructions for use.

Embodiment 25: A kit including: (a) one or more anti-IPF Biomarker agents, wherein the one or more anti-IPF Biomarker agents include an anti-TIMP1 agent, an anti-HA agent, and an anti-PIIINP agent; and (b) instructions for use.

Embodiment 26: The kit of any one of embodiments 23-25, wherein the one or more anti-IPF Biomarker agents include one or more antibody agents.

Embodiment 27. The kit of embodiment 26, wherein one or more of the antibody agents are labeled with a detectable moiety.

Embodiment 28. The kit of embodiments 23-27, further including one or more control samples.

Embodiment 29. The kit of embodiment 28, wherein the control samples include one or more IPF Biomarker standards.

Embodiment 39. Use of a kit according to embodiments 23-29 in an in vitro diagnostic assay to diagnose IPF in a subject.

### Examples

An Example is provided hereinbelow. However, the present disclosure is to be understood to not be limited in its application to the specific experimentation, results, and laboratory procedures disclosed herein after. Rather, the Example is simply provided as one of various embodiments and is meant to be exemplary, not exhaustive.

This Example concerns the measurement of IPF biomarkers in a convenient blood sample to accurately identify (i.e., aid in diagnosis) and potentially monitor the progression of IPF in patients.

The biomarkers tested were tissue metallopeptidase Inhibitor 1 (TIMP1), hyaluronan (HA), and Procollagen Type III N-terminal propeptide (PIIINP). These tests are immunoassays that run on various automated immunoassay platforms (such as, but not limited to, ATELLICA® and ADVIA CENTAUR® Immunoassay Analyzer Systems (Siemens Healthineers, Inc.; Malvern, PA). The biomarkers can be used alone or in combination with one another and/or in combination with other patient clinical data to accurately identify IPF patients.

In this Example, 99 patient serum samples were tested using Siemens TIMP1, HA, and PIIINP immunoassay tests. There were 53 samples from confirmed IPF patients and 46 control patient samples consisting of 23 smokers and 23 non-smokers.

A logistic regression was used to compare biomarkers to predict the probability of IPF versus controls.

The performance results were calculated by doing a ‘leave 10 out’ cross validation, where the logistic model was reconstructed five hundred times. Each time the model was constructed, only 89 out of 99 of the specimens were used to construct the model. The model was then used to predict the remaining 10 specimens. The sensitivity and specificity from comparing these 10 specimens to clinical truth was averaged across all 500 simulations/iterations to arrive at cross-validated performance for sensitivity and specificity.

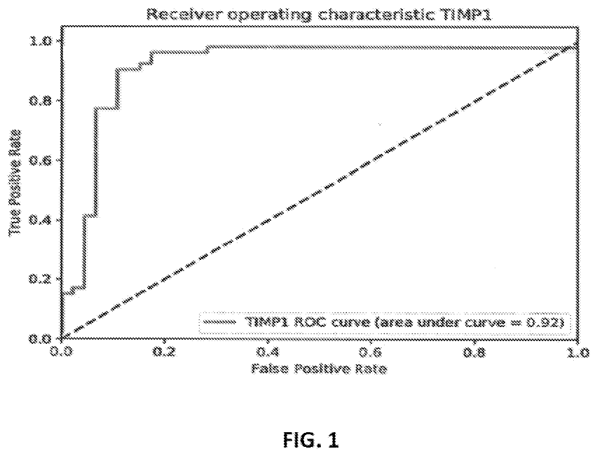

The predictive value of TIMP1 alone to identify IPF was excellent, with an optimum sensitivity of 91.7% and specificity of 87.2%, as shown in the receiving operator curve (ROC) of FIG. 1, and as shown in Table 1 below.

TABLE 1
TIMP1 ROC

| Cut-off Probability | Sensitivity | Specificity |
|---|---|---|
| 90% | 45.5% | 92.9% |
| 70% | 69.7% | 92.9% |
| 50% | 91.7% | 87.2% |
| 45% | 91.3% | 84.0% |
| 40% | 95.3% | 82.1% |
| 30% | 96.5% | 78.5% |
| 20% | 97.7% | 63.2% |
| 10% | 98.2% | 38.2% |
| 5% | 98.0% | 11.8% |

The predictive value of combining TIMP1 and HA with regression analysis further improved the prediction of IPF from controls, as shown in Table 2.

TABLE 2
TIMP1 AND HA ROC

| Cut-off Probability | Sensitivity | Specificity |
|---|---|---|
| 70% | 69.50% | 93.70% |
| 55% | 86.60% | 90.80% |
| 50% | 92.30% | 90.10% |
| 45% | 95.40% | 86.20% |
| 40% | 96.20% | 84.00% |
| 30% | 96.20% | 77.80% |
| 20% | 96.50% | 60.20% |
| 10% | 98.30% | 38.00% |
| 5% | 98.10% | 15.10% |

Therefore, this Example demonstrates the use of TIMP1 alone or in combination with HA in a logistic regression model to reliably predict IPF. The combination of TIMP1, HA, and other clinical data biomarkers (such as, but not limited to, PIIINP) in a logistic regression model provides discriminate scoring systems for more accurate staging and grading of IPF, as well as more accurate monitoring of disease progression and responses to therapies.

Thus, in accordance with the present disclosure, there have been provided compositions, devices, and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

1. A method of determining the presence, severity, and/or predisposition of Idiopathic Pulmonary Fibrosis (IPF) in an individual, the method comprising the steps of:
(a) obtaining a biological fluid sample from an individual;
(b) incubating the biological fluid sample with an antibody that specifically binds to tissue metallopeptidase inhibitor 1 (TIMP1) under conditions that allow for formation of an antibody-TIMP1 immunocomplex;
(c) measuring an amount of antibody-TIMP1 immunocomplex formed to obtained a measured value for TIMP1 in the sample; and
(d) using a mathematical algorithm to obtain an IPF score based on the measured value of TIMP1 in the sample.

2. The method according to claim 1, wherein the biological fluid sample is selected from the group consisting of blood, serum, plasma, saliva, sputum, mucus, nasal, nasopharyngeal, anterior nasal, oropharyngeal, tracheal, bronchoalveolar, and combinations thereof.

3. The method according to claim 1, wherein the IPF score is used to support, predict, or substitute the histological score of a lung biopsy.

4. The method according to claim 1, wherein the mathematical algorithm is a discriminant function algorithm.

5. The method according to claim 1, wherein the discriminant function algorithm is a linear discriminant function algorithm.

6. The method according to claim 1, wherein the IPF score is at least one factor to determine a treatment strategy for the individual.

7. The method according to claim 1, wherein the IPF score is at least one factor used to monitor the efficacy of an implemented treatment strategy for the individual.

8. The method according to claim 1, wherein the IPF score is at least one factor used to determine whether the individual should obtain a lung biopsy.

9. The method according to claim 1, wherein the IPF score is at least one factor used to evaluate the degree of IPF in the individual.

10. A method of determining the presence, severity, and/or predisposition of Idiopathic Pulmonary Fibrosis (IPF) in an individual, the method comprising the steps of:
(a) obtaining a biological fluid sample from an individual;
(b) selecting at least two diagnostic markers of a dynamic process of extracellular matrix synthesis and/or extracellular matrix degradation from said sample, wherein the at least two diagnostic markers are selected from the group consisting of tissue metallopeptidase inhibitor 1 (TIMP1), hyaluronan (HA), and Procollagen Type III N-terminal propeptide (PIIINP);
(c) measuring the amount of each of the at least two diagnostic markers in the sample to obtain a measured value for each of the at least two diagnostic markers; and
(d) combining the measured values of the at least two diagnostic markers using a mathematical algorithm to obtain an IPF score.

11. The method according to claim 10, wherein the at least two diagnostic markers are TIMP1 and HA.

12. The method according to claim 10, wherein the at least two diagnostic markers are TIMP1 and PIIINP.

13. The method according to claim 10, wherein the at least two diagnostic markers comprise TIMP1, HA, and PIIINP, and wherein step (d) is further defined as combining the measured values of the three diagnostic markers using the mathematical algorithm to obtain the IPF score.

14. The method according to claim 10, wherein the IPF score is used to support, predict, or substitute the histological score of a lung biopsy.

15. The method according to claim 10, wherein the mathematical algorithm is a discriminant function algorithm.

16. The method according to claim 15, wherein the discriminant function algorithm is a linear discriminant function algorithm.

17. The method according to claim 10, wherein the IPF score is at least one factor to determine a treatment strategy for the individual.

18. The method according to claim 10, wherein the IPF score is at least one factor used to monitor the efficacy of an implemented treatment strategy for the individual.

19. The method according to claim 10, wherein the IPF score is at least one factor used to determine whether the individual should obtain a lung biopsy.

20. The method according to claim 10, wherein the IPF score is at least one factor used to evaluate the degree of IPF in the individual.

21. A non-transitory computer readable medium containing executable instructions that when executed cause a processor to perform operations comprising the method of claim 1.

22. A composition comprising:
(a) one or more IPF Biomarkers, wherein the one or more IPF Biomarkers comprise:
tissue metallopeptidase inhibitor 1 (TIMP1), hyaluronan (HA), and Procollagen Type III N-terminal propeptide (PIIINP), or a combination thereof; and
(b) one or more anti-IPF Biomarker agents, wherein the one or more anti-IPF Biomarker agents comprise: an anti-TIMP1 agent, an anti-HA agent, an anti-PIIINP agent, or a combination thereof.

23. A kit for detecting IPF, said kit comprising:
(a) one or more anti-IPF Biomarker agents, wherein the one or more anti-IPF Biomarker agents comprise:
(i) an anti-TIMPlagent,
(ii) an anti-HA agent,
(iii) an anti-PIIINP agent,
(iv) a combination thereof; and
(b) instructions for use.

24. A kit for detecting IPF, said kit comprising:
(a) one or more anti-IPF Biomarker agents, wherein the one or more anti-IPF Biomarker agents comprise (i) an anti-TIMP1 agent and an anti-HA agent, (ii) an anti-PIIINP agent and an anti-TIMP1 agent, or (iii) an anti-HA agent, an anti-PIIINP agent, and an anti-TIMP1 agent; and
(b) instructions for use.

25. A kit comprising:
(a) one or more anti-IPF Biomarker agents, wherein the one or more anti-IPF Biomarker agents comprise an anti-TIMP1 agent, an anti-HA agent, and an anti-PIIINP agent; and
(b) instructions for use.

26. The kit of claim 23, wherein the one or more anti-IPF Biomarker agents comprise one or more antibody agents.

27. The kit of claim 26, wherein one or more of the antibody agents are labeled with a detectable moiety.

28. The kit of claim 23, further comprising one or more control samples.

29. The kit of claim 28, wherein the control samples comprise one or more IPF Biomarker standards.

30. Use of a kit according to claim 23 in an in vitro diagnostic assay to diagnose IPF in a subject.


SEQUENCE LISTING (2 sequences)

SEQ ID NO: 1 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 2 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL